(19) United States
(12) Patent Application Publication
(10) Pub. No.: US 2022/0233227 A1
(43) Pub. Date: Jul. 28, 2022

(54) CRYO-ABLATION CATHETER

(71) Applicant: ArtFix LTD, Ariel (IL)

(72) Inventors: Dvir KEREN, Tel Aviv (IL); Lior LEVANONY, Bat Hefer (IL); Oron FELDMAN, Tel Aviv (IL)

(73) Assignee: ArtFix LTD, Ariel (IL)

(21) Appl. No.: 17/615,125

(22) PCT Filed: May 26, 2020
(86) PCT No.: PCT/IL2020/050576

§ 371 Date: Nov. 30, 2021

Related U.S. Application Data
(60) Provisional application No. 62/854,335, filed on May 30, 2019.

(51) Int. Cl. A61B 18/02

(52) U.S. Cl. CPC A61B 18/02; A61B 2018/00577; A61B 2018/0212

(57) ABSTRACT

A cooling frame of a cryoablation catheter has tubing defining at least two extents of cooling tubing each extending between a proximal side and a distal tip of the cooling frame, with a tensioning strut also extending between the proximal side and the distal tip. The tensioning member, in some embodiments, is separately adjustable to press the cooling tubing against a lumenal wall of a body organ targeted for ablation by pressure against an opposite wall. In some embodiments, a loop defined by the cooling tubing is sized to surround all the pulmonary vein ostia of a left atrium, then be chilled by circulation of coolant within the cooling tubing, producing a substantially contiguous loop that electrically isolates the pulmonary vein ostia from the rest of the left atrium.

### RELATED APPLICATIONS

This application claims the benefit of priority under 35 USC § 119(e) of U.S. Provisional Patent Application No. 62/854,335 filed May 30, 2019; the contents of which are incorporated herein by reference in their entirety.

### FIELD AND BACKGROUND OF THE INVENTION

The present invention, in some embodiments thereof, relates to the field of tissue ablation; and more particularly, but not exclusively, to cryoablation of tissue from within the lumenal space of an organ.

Currently, ablation is a gold standard therapy for patients who suffer from atrial fibrillation. While traditionally the ablation was done using RF means, an increasing segment of physicians uses a cryoballoon to achieve ablation. Similar to ablation by RF means, the cryoballoon catheter is inserted via an endovascular approach through the septum (i.e. trans-septally). The physician inflates the cryoballoon individually inside each of the four pulmonary veins, aiming to achieve an ablation in a ring-like geometry, along the connection of the pulmonary vein with the left atrium. The procedure may be repeated for each vein one or more times.

### SUMMARY OF THE INVENTION

There is provided, in accordance with some embodiments of the present disclosure, a cooling frame of a cryoablation catheter comprising: a proximal side; a distal connector, sized to fit within an overtube of a catheter; tubing defining at least one extent of cooling tube configured to be chilled by a cooling flowing therein, and extending between the proximal side and the distal connector region; and a tensioning strut, extending between the proximal side and the distal connector.

In some embodiments, the cooling frame comprises at least a second extent of cooling tube extending between the proximal side and the distal connector region.

In some embodiments, the cooling frame is configured to self-expand from a collapsed configuration sized to fit within the catheter overtube to an expanded configuration.

In some embodiments, the cooling frame comprises at least a second extent of cooling tube extending distally from the distal connector region in the collapsed configuration, and, in a deployed configuration, recurving from the distal connector in a proximal direction back to the proximal side of the cooling frame.

In some embodiments, a deployment length of the tensioning strut is configured to be advanced relative to the catheter overtube separately from the cooling tubing while remaining connected to the cooling tubing at the distal connector.

In some embodiments, the at least two extents of cooling tubing deploy by assuming a curvature that defines an ablation line configured to be brought into contact with a targeted isolation region.

In some embodiments, the ablation line is a loop.

In some embodiments, a main curve of the tensioning strut deploys by radial expansion away from a central proximal-to-distal axis of the cooling frame in a direction away from the ablation line.

In some embodiments, the cooling frame is sized to deploy within a left atrium lumen, with a region of lumenal wall comprising the pulmonary vein ostia located between contacts of the two extents of cooling tubing with lumenal tissue of the left atrium, and the tensioning strut positioned radially opposite the region of lumenal wall comprising the pulmonary vein ostia.

In some embodiments, the main curve of the tensioning strut has an anisotropic cross-section at least 1.5× longer in a first direction than in a direction perpendicular to the first direction.

In some embodiments, the cross-section is rectangular.

In some embodiments, the cross-section is oval.

In some embodiments, the main curve expands to lie within a plane.

In some embodiments, the tensioning strut comprises a secondary curve, curving in a direction opposite the main curve.

In some embodiments, the secondary curve and the main curve lie substantially within a single plane.

In some embodiments, the main curve extends at least 70% of the way between the proximal side and the distal connector, when the cooling frame is deployed, and the secondary curve extends the remainder of the way to the distal tip.

In some embodiments, the wherein each of the at least one extents and the tensioning strut connect to a proximal side of the distal tip.

In some embodiments, the distal connector is a distal tip of the cooling frame.

In some embodiments, the tubing comprises nitinol tubing.

In some embodiments, the tensioning strut comprises a nitinol alloy.

In some embodiments, the cooling frame comprises at least one coolant delivery tube, positioned in fluid communication with a lumen of the tubing, and configured to deliver coolant to the lumen.

In some embodiments, a supply port of the coolant delivery tube is configured to move within the lumen of the tubing.

In some embodiments, the at least one coolant delivery tube comprises a plurality of supply ports configured to delivery coolant to the lumen.

In some embodiments, the cooling frame is configured with a lumenal region between the coolant deliver tube and the cooling tube, allowing return of coolant proximally past the coolant delivery tube, thereby creating a counter-cooling effect.

In some embodiments, the distal connector comprises a swivel joint.

In some embodiments, the swivel joint is configured to allow rotation of a distal portion of the cooling tube relative to the tensioning strut within a plane of a first rotational axis, whereby the cooling tube assumes a curved shape upon deployment.

In some embodiments, the swivel joint is configured to allow rotation of a distal portion of the cooling tube relative to the tensioning strut around a second rotational axis, whereby the curved shape of the cooling tube is rotatable to a plurality of positions while the tensioning strut remains in place.

In some embodiments, the cooling frame comprises a plurality of tensioning struts extending between the proximal side and the distal connector.

In some embodiments, in the collapsed configuration, the tensioning strut extends distally from the distal connector, and upon expansion to a deployed state, re-curves proximally to the proximal side.

In some embodiments, the tensioning strut is joined to the proximal side by a shaping member which can be shortened to secure the tensioning strut at the proximal side.

In some embodiments, the at least two extents of tubing comprise a plurality of tubing pieces, each terminating distally at the distal connector, and the distal connector is a distal tip.

In some embodiments, the distal connector connects lumens of the tubing pieces through an interconnecting lumen of the distal connector.

In some embodiments, the distal tip comprises a cap covered by a hollow tip, and the interconnecting lumen is defined within the cap and the hollow tip.

In some embodiments, the distal connector comprises mutually attached connecting tubes, into which the tubing and tensioning strut are inserted.

In some embodiments, the tubing and tensioning strut connect to the distal connector through a proximal side.

There is provided, in accordance with some embodiments of the present disclosure, a method of manufacturing a hollow distal tip of a cooling frame of a cryoablation catheter, the method comprising: inserting distal ends of at least one tubing section into a sleeve assembly; inserting the sleeve assembly into a cap; and placing a hollow tip over the cap; wherein the tubing sections are attached to the sleeve assembly by crimping, and the sleeve assembly is attached to the cap by an adhesive.

There is provided, in accordance with some embodiments of the present disclosure, a hollow distal tip of a cooling frame of a cryoablation catheter, comprising: a sleeve assembly, sized to accept a distal end of at one tubing section; a cap into which the sleeve assembly is inserted; and a hollow tip over the cap; wherein sleeve assembly attaches to the distal end by crimping, and to the cap by an adhesive.

There is provided, in accordance with some embodiments of the present disclosure, a method of cryoablation, comprising: deploying a tube of a cryoablation frame from a catheter; curving the tube elastically to contact and conform to a lumenal surface of a heart left atrium, while a strut of the cryoablation frame forces the tube against the lumenal surface; and circulating coolant into the tube while it remains in contact with the lumenal surface, thereby creating an ablation in the lumenal surface that surrounds all the pulmonary ostia of the heart left atrium.

There is provided, in accordance with some embodiments of the present disclosure, a method of cryoablation comprising: deploying a cryoablation frame comprising a superelastic metal alloy from a catheter into contact with the lumenal surface of a beating heart left atrium; circulating coolant into tubes of the frame, thereby cooling the superelastic metal alloy enough to reduce its elasticity by at least 50%; and adhering cooled tubes of the frame to the surface of the heart left atrium, by freezing, thereby maintaining thermal contact with the surface.

In some embodiments, the superelastic metal alloy comprises nitinol.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A schematically illustrates a deployed cooling frame of a cryoablation catheter, according to some embodiments of the present disclosure;

FIG. 1B schematically illustrates cooling frame retracted into overtube of a cryoablation catheter, according to some embodiments of the present disclosure;

FIG. 1C is a block diagram schematically illustrating a catheter system for cryoablation using cooling frame, according to some embodiments of the present disclosure;

FIG. 2 is a schematic flowchart of a method of operating the cooling frame of FIGS. 1A-1B, according to some embodiments of the present disclosure;

FIGS. 3A-3D schematically illustrate a deployment sequence of for deployment of cooling frame within a left atrium, according to some embodiments of the present disclosure;

FIGS. 4A-4B schematically illustrate selected phases of the deployment of cooling frame within a left atrium, according to some embodiments of the present disclosure;

FIGS. 4C-4D schematically illustrate expansion states of cooling frame during deployment, corresponding to the in situ states described in relation to FIGS. 4A and 4B, respectively, according to some embodiments of the present disclosure;

FIGS. 5A-5B schematically illustrate different positions of a coolant supply tube within a cooling tube of cooling frame, according to some embodiments of the present disclosure;

FIG. 5C is a schematic flowchart of a method of delivering coolant to cooling frame, according to some embodiments of the present disclosure;

FIG. 5D schematically illustrates a two-tube arrangement for coolant supply, according to some embodiments of the present disclosure;

FIG. 6 is a schematic flowchart of a method of maintaining contact of a cooling frame with a heart during operation, according to some embodiments of the present disclosure;

FIG. 7 schematically illustrates a cutaway view of a channeled frame connector at a distal tip of cooling frame, according to some embodiments of the present disclosure;

FIGS. 8A-8F illustrate stages in the manufacture of a frame connector placed at a distal tip of a cooling frame, according to some embodiments of the present disclosure;

FIGS. 9A-9E represent different methods of circulating cooling fluid within a cooling frame, according to some embodiments of the present disclosure;

FIG. 10 schematically represents a cooling frame of a cryoablation catheter comprising a redoubling cooling tube, according to some embodiments of the present disclosure;

FIG. 11 schematically represents a cooling frame of a cryoablation catheter comprising a redoubling cooling tube and a tensioning member, according to some embodiments of the present disclosure;

FIGS. 12A-12B schematically illustrate a cooling frame comprising a single lumen-spanning arc of a single cooling tube, according to some embodiments of the present disclosure;

FIG. 13 schematically illustrates a cooling frame comprising two separate lumen-spanning arcs comprising cooling tubes, respectively, according to some embodiments of the present disclosure;

FIG. 14 schematically illustrates a cooling frame comprising two separate lumen-spanning arcs comprising cooling tubes, respectively, each having its own tensioning element, according to some embodiments of the present disclosure;

FIGS. 15A-15B schematically illustrate a cooling frame comprising at least one shaping member, which is operable to pull a free distal end of a cooling tube and/or of an extension of the cooling tube, back toward a proximal region of the cooling frame; and

FIGS. 16A-16B, 17A-17C and 18A-18C schematically illustrate a cooling frame comprising a swiveling distal connection, according to some embodiments of the present disclosure.

### DESCRIPTION OF SPECIFIC EMBODIMENTS OF THE INVENTION

The present invention, in some embodiments thereof, relates to the field of tissue ablation and more particularly, to cryoablation of tissue from within the lumenal space of an organ.

### Overview

A broad aspect of some embodiments of the present disclosure relates to a cryoablation device configured for ablating tissue along a path from within a body lumen.

In some embodiments, the cryoablation device is used for ablation treatment of atrial fibrillation. One ablation pattern considered potentially effective preferably comprises creation of a continuous, substantially unbroken ring of ablated tissue which surrounds the ostia of the pulmonary veins, thereby isolating the rest of the atrium from, e.g., re-entrant electrical conduction from the pulmonary veins. In some embodiments of the present invention, a cooling frame is provided which deploys from a catheter-delivered configuration to a state which is sufficiently expanded, strong, and stable that it potentially ensures contacts with cooling surfaces of the device to allow creating lesions which result in effective treatment. In some embodiments, frame strength and stability is achieved substantially without interfering with blood flow; i.e., without use of a balloon.

In some embodiments, the cooling frame combines at least one cooling tube, used to perform cryoablation, and a tensioning device, which helps to ensure that the at least one cooling tube establishes a reliable and reproducible contact with endoluminal surfaces targeted for ablation. Structural and/or cooling components of the frame, in some embodiments, comprises a superelastic material such as nitinol, which potentially gives added reliability for the device to reach and maintain an expanded state without support by pressurized inflation (e.g., of a balloon), potentially to the degree of stretching targeted tissue over the frame for enhanced security of contact.

In some embodiments, continuity of the ablation is achieved by ablating from an expanding loop that contacts the whole ablation region simultaneously while cooling it. In some embodiments, continuity of ablation is achieved with stabilization of a portion of the frame by expanding it into place with the targeted body lumen, then moving a cooling tube relative to the rest of the frame in order to ablate at two or more locations in reliably selected relative locations.

In some embodiments, the device is delivered over a catheter configured for use in ablating within a left heart atrium. The catheter is provided with the distally deployable cooling frame configured for cryoablation of tissue near and/or surrounding the pulmonary vein ostia; optionally all pulmonary vein ostia at once (typically four in number, and varying in the healthy population between three and five pulmonary veins).

For example, the catheter is inserted into the left atrium in a conventional endovascular transseptal approach. Once the cooling frame is placed inside the left atrium, in some embodiments, the physician retracts a catheter-external sleeve (i.e., an overtube). This allows self-deploying of a cooling frame. Additionally or alternatively, the cooling frame is extruded from the catheter into the left atrium.

After the cooling frame is placed, the physician activates a coolant flow through the tubes (e.g., a flow of pressurized nitrogen). The cooling frame cools tissue it contacts, ablating it.

To end the procedure, the physician retracts the cooling frame into the sheath, collapsing it, and retracts the system through the guiding catheter.

Potential advantages of ablating around all pulmonary veins simultaneously include:

The shape of ablations formed mimic ablations of an open heart surgery technique (the Maze procedure), known to be very effective, but now relatively disused due to its invasiveness.

Ablation of the four pulmonary vessels simultaneously instead of each one individually potentially shortens and/or simplifies the ablation procedure.

Ablation within the left atrium is optionally performed without blocking blood flow, e.g., in contrast to the operation of certain balloon ablation devices.

Potential problems of ablation using a frame that contacts a lumenal surface over a substantial extent of the surface (e.g., circumscribing the ostia of all pulmonary veins, and/or extending between a septal wall of a left atrium and the left atrial wall opposite the septal wall) include readily obtaining stable and reliable surface contact all around a targeted ablation pathway, using a transcatheter delivered device.

An aspect of some embodiments of the invention relates to a cooling frame comprising one or more cooling tubes, and a tensioning member actuatable to press the cooling tube(s) against the inner surface of a body lumen within which cryoablation is being performed.

In some embodiments, the cooling tube(s) and/or the tensioning member comprise a shape memory and/or superelastic alloy such as nitinol. Superelasticity comprises an elastic response to applied stress, related to reversible movements during phase transformation of a crystal; e.g., between the austenitic and martensitic phases of the crystal. Shape memory is a related property that allows a deformed alloy to be returned to an original set shape by a change in conditions (e.g., upon heating).

The cooling frame, in some embodiments, comprises two main components:

One or more cooling tubes, which deploy to conform to a body lumen interior, thereby defining an ablation surface targeted for ablation (e.g., creating a substantially closed-loop geometry sized to surrounding the pulmonary vein ostia).

A tensioning member. In some embodiments, the tensioning member comprises a strut that deploys to a curve. The curve may be to an opposite direction from a curve of the cooling tube(s). The curve may be opposite the direction of the lumenal surface targeted for ablation, and/or radially opposite a loop surface or other ablation surface defined by the cooling tube(s) which contacts the lumenal surface targeted for ablation. In some embodiments, the tensioning member comprises a plurality of members which expand in two or more directions to position and stabilize the frame. By pressing against portions of the lumen (e.g., an atrial wall opposite the pulmonary veins), the tensioning member potentially acts to help ensure that the lumenal surface targeted for ablation makes reliable surface contact with the cooling tube(s).

In some embodiments, the tensioning member spans the cooling frame between a proximal side and a distal side of the cooling frame. Preferably, the tensioning member is physically coupled to the cooling tubes at a both the proximal side and the distal side in order to stabilize their shape and/or positioning.

In some embodiments, reliably establishing and maintaining surface contact is assisted by the use of planar curves to define the cooling frame. Planar curves have the potential advantage of being relatively resistant to the transmission of deformation into out-of-plane directions, particularly if the planarity of the curves is furthermore supported by at least one member having a cross-section which is anisotropic or “ribbon-like”—that is, wider in one direction than in another (e.g., by a factor of at least 1.5, 2, 3, 4 or another factor). Even one such anisotropic member (e.g., the tensioning member) potentially is sufficient to stabilize the whole device against torqueing, since one member that resists twisting acts to resist twisting of the device overall.

A potential advantage of the tube(s)-and-tensioning member cooling frame design is simple and reliable control. For example, a cryoballoon' s pressure (one of the factors which can affect its tissue contact) is potentially temperature dependent to a significant degree (e.g., due to thermodynamic laws governing gas volume as a function of temperature). By relying on elastic tension rather than gas pressure, cooling control and contact control are potentially decoupled.

An aspect of some embodiments of the invention relates to continued stabilization of device-tissue contact as the superelasticity of the cooling tube(s) reduces during cryoablation.

In some embodiments, a method of cryoablation comprises expanding a frame comprising a plurality of tubes and/or struts to press against a cryoablation target, and cooling one or more of the tubes and/or struts to a temperature at which their elasticity is reduced (for example, reduced by at least 50%), while at least one tube and/or strut remains uncooled, with elasticity maintained (for example, maintained to at least 95% of its original elasticity). In some embodiments, the uncooled tube and/or strut is provided with an anisotropic cross-section (e.g., at least 1.5, 2, 3 or more times wider in one direction than in an orthogonal direction).

A potential advantage of providing a tensioning member separate from the cooling tube(s) is to protect against changes in elasticity (e.g., reduction of superelasticity) as a function of temperature. In some embodiments, the superelasticity and/or planar stability of the tensioning member is sufficient to stabilize the cooling frame even as superelasticity of the cooling tube(s) is reduced as they near cryoablation temperatures. In some embodiments, at least a portion of stability reduction due to loss of superelasticity at cryotemperatures is compensated for (and potentially even improved upon) by freeze-adherence of the cooling tube(s) to contacted tissue.

An aspect of some embodiments of the invention relates to the construction of a distal connector (e.g., a distal tip) of the cooling frame. The distal connector has at least two important functions:

It allows the tensioning member to recontact the cooling tubes(s) at a distal position, so that it can provide support at both distal and proximal sides of the frame;

Before deployment, it joins tensioning member and cooling tube(s) in a way which can be collapsed to a small-diameter package without stressing any element to the point of breaking.

A third function, in some embodiments—especially when the distal connector is also a distal tip—is to join the tensioning member to the cooling tube(s) in a structure which is atraumatic to the extent that it will not cause injury to the lumen in which it deploys by poking, cutting, or scraping.

In some embodiments, the cooling frame comprises three or more tube and/or strut members, each of which extends from a proximal side of the frame to connect with a distal tip of the cooling frame, and each connecting to the cooling frame from a same proximal side of the distal tip. In some embodiments, a segment of the cooling tube projects past the distal connector, curving in a new direction (e.g., recurving proximally again) to form a second ablation segment of the cooling frame.

In some embodiments, the cooling frame is configured to reversibly convert between a collapsed state and an expanded state. In the collapsed state, each of the tube and/or strut members extend substantially parallel to one another, optionally connecting to the distal tip without creating a region of side protrusion (e.g., a region extending beyond the perimeter of the tip cross-section relative to a longitudinal axis of the collapsed cooling frame).

In some embodiments, in the expanded state, a midline of each of the tube and/or strut members extends through a different plane (optionally a best-fitting plane) than any of the other tube and/or strut members.

In some embodiments, the tip is re-oriented by expansion of the cooling frame so that it points substantially sideways (or optionally even partially proximally), relative to the initial direction of distal extension of the device. This potentially helps to ensure that the tip does not interfere with contact of cooling tube(s) of the cooling with targeted ablation surfaces.

The deployed three strut and/or tube members, in some embodiments, are arrayed at least partially in opposition around the cooling frame perimeter, while the tip comes to occupy a sideways orientation—and, optionally, all these members nevertheless connect to the tip on its proximal side. Correspondingly, in some embodiments, at least one of the strut and/or tube members has a deployed main curve that in some region extends proximally (points backwards) relative to the (sideways-deployed) distal tip in order to reach the proximal side of the distal tip where it is connected. To finally connect, a secondary curve is optionally provided to this member that turns the member back to extend in the tip-relative distal direction.

In some embodiments, the secondary curve is provided to a strut that acts as a tensioning member. In some embodiments, the secondary curve provides an additional function in taking up any excess extent of the tensioning member, beyond that needed to press the cooling frame fully into place. This provides a potential safety mechanism by preventing advance of the tensioning member from stretching the lumenal tissue (e.g., of the left atrium) to the point of rupture or other damage. In some embodiments, both the main curve and the secondary curve lie substantially in a same plane through which centerlines of the curve extend.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Exemplary Embodiment of a Cooling Frame

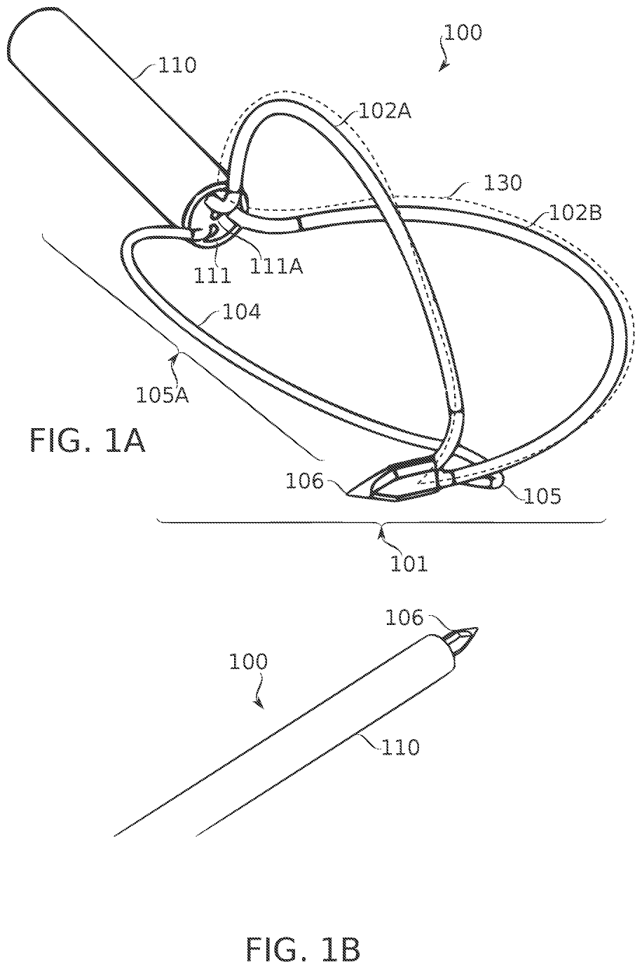

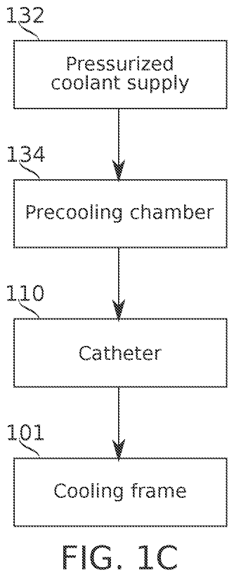

Reference is now made to FIG. 1A, which schematically illustrates a deployed cooling frame 101 of a cryoablation catheter 100, according to some embodiments of the present disclosure. Reference is also made to FIG. 1B, which schematically illustrates cooling frame 101 retracted into overtube 110 of a cryoablation catheter 100, according to some embodiments of the present disclosure. Further reference is made to FIG. 1C, which is a block diagram schematically illustrating a catheter system for cryoablation using cooling frame 101, according to some embodiments of the present disclosure.

In some embodiments, a cryoablation catheter 100 comprises an overtube 110, within which (FIG. 1A) a cooling frame 101 is deliverable, e.g., via blood vessels, to a target organ lumen such as a heart left atrium. Upon reaching its target, cooling frame 101 is deployable to an expanded state (FIG. 1A) used for the ablation itself.

In some embodiments, the cooling frame 101 of cryoablation catheter 100 comprises at least one cooling tube 102A, 102B, optionally arranged, upon expansion, to define a contact surface (e.g., extending underneath the dotted line of loop 130), shaped to be pressed into contact with tissue of the curved interior surface of the target organ lumen. In some embodiments, the line of contact comprises a substantially loop-shaped region of contact that surrounds a region which is to be isolated (e.g., electrically isolated) from the rest of the lumen by cryoablation. Herein this region is referred to as a targeted isolation region.

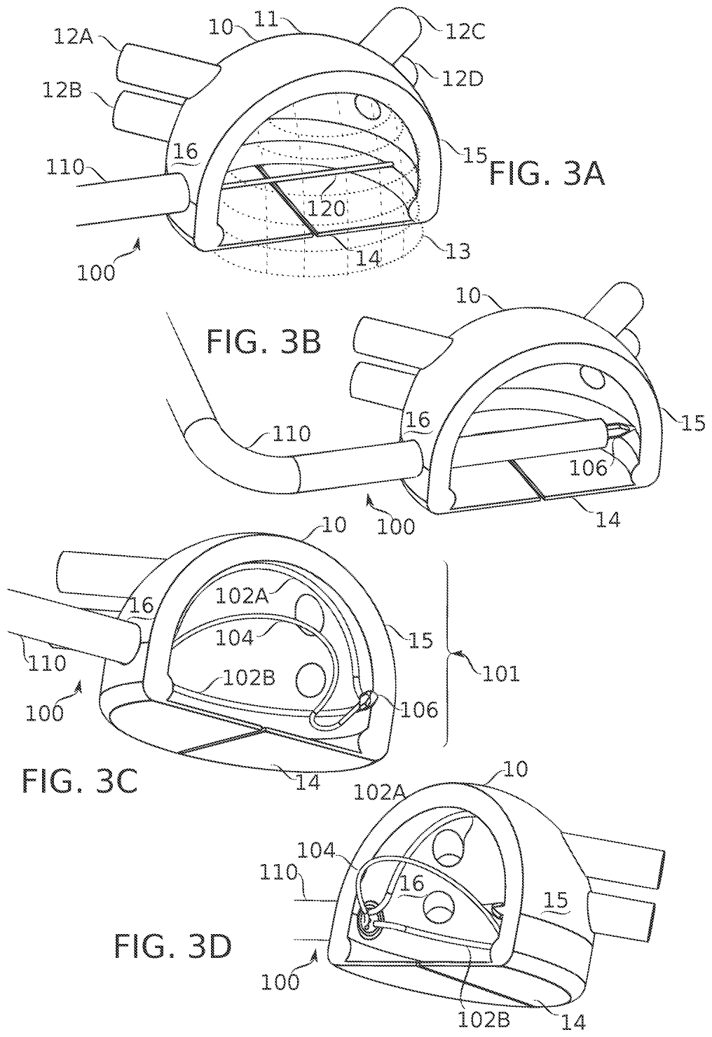

Optionally, upon circulation of cooling fluid through cooling tubes 102A, 102B, contacted tissue is ablated by cooling to temperatures that result in cellular death. In some embodiments, the size of loop 130 is large enough to encompass a plurality of left pulmonary vein ostia, for example as discussed herein in relation to in situ deployment of the cooling frame 101 and FIGS. 3A-3D. In some embodiments, cooling tubes 102A, 102B exit overtube 110 closely enough to one another, and/or meet closely enough at tip 106, that the spread of the cryogenic ablating zone from each tube (e.g., to a range of about ±5 mm, or another distance) results in closure of loop 130. In some embodiments, tip 106 itself becomes cryogenically cold sufficiently during operation of the device that it also acts as an ablating surface. For example, it cools due to contact with cooling tubes 102A, 102B, and/or itself comprises a passageway for cryogenic coolant.

In some embodiments, cooling tubes 102A, 102B comprise a plurality of tubes which are joined together proximally at base 111, and distally at tip 106. In some embodiments, tip 106 includes a lumen that joins tubes 102A, 102B so that coolant fluid is allowed to circulate between cooling tubes 102A, 102B. Alternatively, in some embodiments, tubes 102A, 102B terminate blindly at tip 106, and are cooled separately. In some embodiments, cooling tubes 102A, 102B together comprise a single extent of tubing with a sharp bend at it distal end in the region of tip 106. However, tight curvature constraints in the region of tip 106 (e.g., to allow retraction of cooling frame 101 into overtube 110) make manufacture of a one-piece tube design potentially more difficult, so that joining separate tubes 102A, 102B at a distal tip 106 has potential advantages.

In some embodiments, cooling tubes 102A, 102B have an outer diameter of about 0.8-2 mm, and a wall thickness of about 100 μm. Alternatively, a non-circular cross-section is used, for example, oval and/or a flat sided cross section (e.g., square, triangular, or another cross-section). A potential advantage of a non-circular cross-section is to increase tissue contact along a flat or flattened side of cooling tubes 102A, 102B.

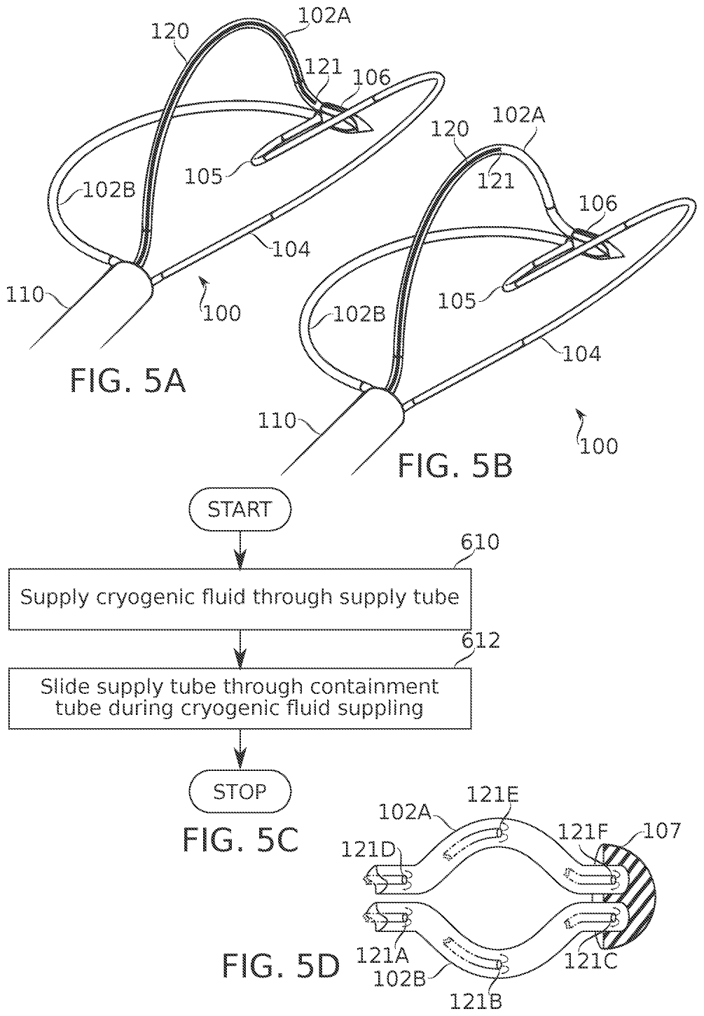

In some embodiments, overtube 110 comprise a polymer, e.g., PTFE and/or nylon. In some embodiments, coolant supply tubing (described as coolant supply tube 120 in relation, for example, to FIGS. 5A-5B) is provided which is positioned and/or positionable inside cooling tubes 102A, 102B; for example as described in relation to FIGS. 5A-5D and/or 9A-9E, herein. Coolant is supplied, e.g., from a pressurized coolant supply 132, optionally via a pre-cooling chamber 132 which reduces the temperature of supplied coolant before it passes through overtube 110 along coolant supply tube 120 and into cooling frame 101. In some embodiments, one or more of pressurized coolant supply 132, overtube 110, and cooling frame 101 comprises one or more sensors. Sensors optionally comprise, for example:

A pressure sensor configured to detect coolant pressure, and to provide pressure data optionally as a basis for controlling coolant flow and/or verifying safe operation.

A temperature sensor, configured to detect temperature at or near cooling frame 101, and to provide temperature data optionally as a basis for controlling coolant flow, coolant supply tube position, and/or verifying safe and/or effective operation.

An electrode, configured to deploy within the region

One or more electrical sensors (electrodes, for example), configured, e.g., to verify tissue contact, for example by using impedance measurements.

One or more electrodes which deploy between cooling tubes 102A, 102B at positions suitable for sensing of myocardial electrical activity, e.g., electrical activity transmitted from outside of loop 130. Such electrodes are optionally used in the verification of ablation effectiveness.

In some embodiments, cooling tubes 102A, 102B divide the cooling section of cooling frame 101 into a respective plurality of separate arc-shaped regions (one for each tube). Mechanically, this has potential advantages for helping to ensure good lumen surface contact along all or most of loop 130. These advantages may be understood, without commitment to a particular theory of operation, as comprising two main factors.

First, corresponding ends (distal ends and proximal ends being understood to correspond) of each of cooling tubes 102A, 102B proceed from almost the same place—one where they separate from each other at base 111 (which can be positioned near the exit from overtube 110); and one where they rejoin at tip 106. Assuming these two positions can be reliably defined at positions in contact with the lumenal wall (this is further described, for example, in relation to FIGS. 3A-3D, herein); then the problem of ensuring good contact along the whole of each tube 102A, 102B is reduced, in some embodiments, to the problem of ensuring good contact along a single planar arc in between two well-defined contact points.

Second, insofar as the two arcs are substantially planar and/or lack an interconnecting gradual curvature, there is potentially an advantage for stiffness. For example, a likelihood is potentially reduced that pushing in one direction will lead to the transmission of frame distortions by torqueing, and/or “sliding” through the curvature into the orthogonal direction.

Stiffness provides a potential advantage, insofar as it provides the arcs of the cooling tubes 102A, 102B with mechanical strength to “stretch out” tissue of the lumen to conform to its arc when pressed against it, in increased preference to relieving force by cooling tube deformation.

There is also a potential advantage, in some embodiments, for activating cooling of the cooling tubes 102A, 102B separately, optionally at different times. For example, this may help to resist instability of the frame due to loss of superelasticity at cryogenic temperatures. Optionally, cooling tubes 102A, 102B are composed of a superelastic alloy. Nitinol, for example, is optionally used; a material well-known for its superelasticity (as well as shape memory) properties.

Shape memory provides a potential advantage for delivery and deployment, allowing flattened (collapsed) delivery packaging of tubes 102A, 102B, and then recovery of a curved shape, without introducing permanent deformation that could interfere with the shape to which they deploy. Superelasticity furthermore potentially assists in deployment of cooling frame 101 to exert force on the lumen in which it is deployed in a way that results in a region of continuous contact.

It should be understood, however, that for any given alloy composition (particularly when restricted to alloys well-accepted for their biocompatibility), superelasticity properties are typically exhibited in relatively narrow temperature ranges. The range can be varied according to the formation of the alloy, but not necessarily with full superelasticity and/or biocompatibility available for all temperatures.

Thus, nitinol alloys capable of exhibiting superelasticity at body temperature are potentially substantially inelastic (in particular, soft and easily deformed) at typical cryoablation temperatures (e.g., temperatures typically well below −20° C.), though the shape memory effect allows them to recover their shapes upon re-warming. Even if some low-temperature superelasticity is retained, there may still be loss of structural strength. This raises a potential problem—particularly in a beating heart chamber—for maintaining cryoablation contact at low temperatures, as superelasticity is lost. While some nitinol alloys exhibit superelasticity at nearer to cryotemperatures, they are potentially more expensive and/or difficult to use in manufacturing.

Accordingly, there arises a potential problem wherein cooling sufficient to cause tissue ablation also impairs superelastic properties of the cooling tubes 102A, 102B which initially help to ensure adequate stability and surface contact with the lumen tissue targeted for ablation. In some embodiments, this problem is addressed in part, through a use of an auxiliary tensioning member 104.

In some embodiments, pressing contact between the cooling tubes 102A, 102B and the interior surface of the target organ lumen is assisted by tensioning member 104, also optionally made of a superelastic material such as nitinol. Tensioning member 104 is not subject to cryoablation temperatures during operation. Optionally, tensioning member 104 is shaped (e.g., shape set during manufacture at a temperature of several hundred ° C.) to deploy as a curving wire. Upon deployment, tensioning member 104 extends distally from the position where cooling frame 101 exits overtube 110 to connect at a distal tip 106 which also is connected with cooling tubes 102A, 102B.

By means of tensioning member 104 (which does not circulate coolant), it is potentially ensured that distal tip 106 remains pressed against the lumenal wall even as cooling tubes 102A, 102B potentially lose superelasticity as a result of reaching cryotemperatures. In some embodiments, loss of superelasticity is compensated for in part by freeze-attachment of the cooling tubes 102A, 102B to tissue as they reach cryotemperatures. In some embodiments, the exchange of contact-maintenance mechanisms occur while the cooling tubes 102A, 102B remain substantially self-supporting. Optionally, exchange of contact-maintenance mechanisms is potentially assisted by the force exerted by tensioning member 104 to keep distal tip 106 pressed against the lumenal wall.

Optionally, control of the position of coolant supply (e.g., by movement of coolant supply tubes within cooling tubes 102A, 102B) assists in managing where and when superelasticity is lost, so that a reliable exchange between tension-based and attachment-based surface contact mechanisms is achieved, for example as described in relation to FIGS. 5A-5D.

In some embodiments, tensioning member 104 comprises a main curve 105A. During deployment, distal advance of tensioning member 104 is controllable separately from cooling tubes 102A, 102B. As more of tensioning member 104 is extruded from overtube 110, main curve 105A assumes an increasingly large bulge, expanding in a radial direction away from a central proximal-to-distal axis of the frame, and reaching a size that causes it contact and exert force against a wall of the target organ lumen that potentially helps press cooling tubes 102A, 102B against another wall section of the target organ lumen (e.g., an opposite wall section). In some embodiments, the unconstrained curvature of main curve 105A has a significantly larger radius of curvature than it will assume in the deployed (and lumen-restricted) form it assumes within a target body lumen (e.g., at least a 50% larger radius of curvature, or larger). In some embodiments, a minimal unconstrained curvature (e.g., 10 mm or less of bending over its whole length) is set on the main curve: just enough to ensure that it will bend in the correct direction upon deployment. This potentially helps to ensure that the main curve will be exerting a higher pressing force when deployed within the constraints of a target lumen.

In some embodiments, main curve 105A comprises a cross section which is wider in one direction that another (e.g., rectangular or oval). This potentially helps to ensure that tensioning member 104 bends within a predetermined plane (e.g., a plane perpendicular to the wider direction of the cross-section).

Optionally, tensioning member 104 also comprises a secondary curve 105. A role of secondary curve 105, in some embodiments, is to re-orient the direction of tensioning member 104 after passing through secondary curve 105A so that it enters distal tip 106 in a direction parallel to (and from the same side as) cooling tubes 102A, 102B. When collapsed, it is a potential advantage for each of tension member 104 and cooling tubes 102A, 102B enter to distal tip 106 from a same (proximal) side, allowing a smaller device diameter in the collapsed state. Optionally, secondary curve 105 comprises the same cross-section as main curve 105A, and optionally secondary curve 105 bends within the same plane as main curve 105A.

In some embodiments, secondary curve 105 is shaped to accept deformation as expanding tensioning member 104 encounters expansion resistance (e.g., due to wall contacts made by cooling frame 101), thereby acting as a strain relief on main curve 105A. This potentially increases the reliable range of advance with which tensioning member 104 can be distally deployed. For example, strain relief from secondary curve 105 potentially reduces a risk of damage to the lumenal wall, and/or a risk of uncontrolled buckling of the device.

Optionally, secondary curve 105 is narrower that main curve 105A (e.g., comprises a region in which tensioning member 104 has a radius of curvature narrower than in main curve 105A, for example, by a factor of 1.5, 2, 3, 4, or another factor). In some embodiments, main curve 105A extends (when deployed) at least 70% or 80% of the distance between a proximal side of the cooling frame 101 and a distal tip 106.

In some embodiments, tensioning member 104 is configured so that main curve 105A extends approximately within a plane that bisects the loop 130 defined by cooling tubes 102A, 102B at about equal distances from each cooling tube 102A, 102B. In some embodiments, this comprises orienting a longer side of a cross-section of tensioning member 104 (e.g., at positions where it is joined to the cooling frame 101) to be substantially perpendicular to the bisecting plane.

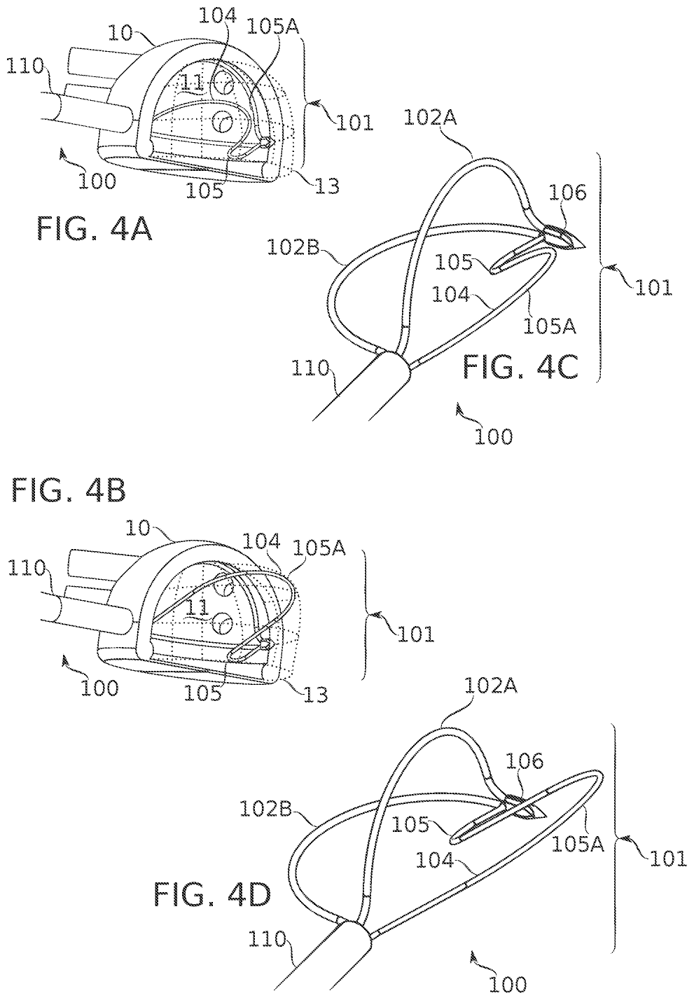

This potentially allows main curve 105A to act simultaneously to press both cooling tubes 102A, 102B about equally against an organ's internal lumenal wall. Additionally or alternatively, in some embodiments, secondary curve 105 is positioned so that upon deployment it also presses against portions of the body organ lumen in which it is deployed. For example, secondary curve 105 presses with a surface outer to its curve against portions of the atrium above a mitral valve 14 (e.g., as shown in FIGS. 4B and 4D, herein). This potentially contributes to tensioning forces.

Optionally, secondary curve 105 is oppositely curved to main curve 1045A, e.g., so that tensioning member 104 forms an “S” shaped curve (albeit with one curve of the “S” potentially being smaller than the other). Optionally, secondary curve 105 is located on a distal side of tensioning member 104, for example as shown in FIG. 1A. In some embodiments, secondary curve 105 is located on a proximal side of tensioning member 104. Optionally, one or more secondary curves 105 are superimposed on the main curve 105A, e.g., forming a sinusoidal or other repeating pattern superimposed on the longer (higher radius) curvature of main curve 105A. In some embodiments, main curve 105A and secondary curve 105 are within a same plane. In some embodiments, they form arcs within separate planes. In some embodiments, one or both of the curves are themselves non-planar.

Optionally, additional tensioning members 104 are provided, for example, extending alongside and/or attached at intervals inside the curves of cooling tubes 102A, 102B. These provide a potential advantage of additional—and optionally more direct—support of cooling tubes 102A, 102B, particularly during periods of cooling below their superelasticity temperature. Constraints on the size of collapsed delivery size of cooling frame 101, however, potentially limit the amount of auxiliary support which can practically be provided. The three-member frame design (two cooling tubes 102A, 102B, and one tensioning member 104) is potentially sufficient.

In some embodiments, base 111 comprises an aperture 111A, sized for passing a guidewire therethrough.

### Deployment and Operation of a Cooling Frame

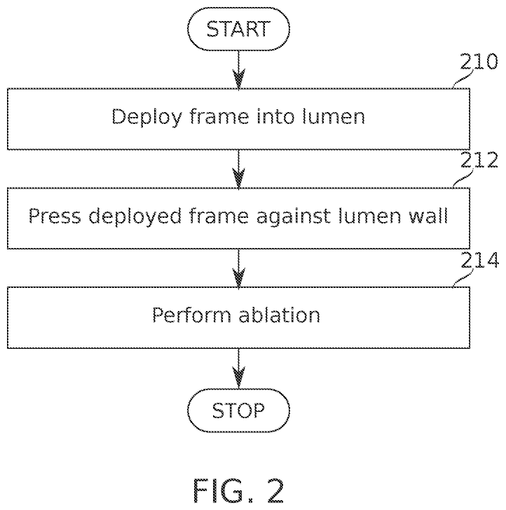

Reference is now made to FIG. 2, which is a schematic flowchart of a method of operating the cooling frame 101 of FIGS. 1A-1B, according to some embodiments of the present disclosure. Further reference is made to FIGS. 3A-3D, which schematically illustrate a deployment sequence of for deployment of cooling frame 101 within a left atrium 10, according to some embodiments of the present disclosure.

In some embodiments, reaching the body lumen is performed with the assistance of a guidewire 120, for example as shown in FIG. 3A. Optionally, access to the left atrium is transseptal. In case another access is used, the orientation of the members of cooling frame relative to overtube 110 is optionally adjusted. Transseptal access provides a potential advantage by creating a device 100 axis extending across left atrium 10 with the distal side of cooling frame 101 in contact with one wall (distal wall 15), and the proximal side of cooling frame 101 readily placed in proximity to the septal wall 16.

Also shown in FIG. 3A are dorsal left atrial wall 11, which optionally includes the ostia of four pulmonary veins 12A, 12B, 12C, 12D (depending on details of patient anatomy, details of the number and arrangement of pulmonary vein ostia may be different). The general location of ventral left atrial wall 13 is indicated with dotted lines; it has been cut away in FIGS. 3A-3D in order to allow viewing of details of device deployment. Mitral valve 14 is schematically indicated as the floor of left atrium 10.

At block 210 (FIG. 2), in some embodiments, a cooling frame 101 is deployed into the body lumen from within which a cryoablation procedure is to be performed, e.g., left atrium 10. For example (FIG. 3B), the overtube 110 is inserted over the guidewire across septal wall 16 and advanced to distal wall 15. Then, optionally, overtube 110 is withdrawn, allowing cooling frame 101 to expand. Optionally, cooling frame 101 is extruded into left atrium 10 from the distal end of overtube 110 from a more proximal position in the left atrium. The advance-then-withdraw sequence illustrated has a potential advantage of avoiding a chance for the expanding frame to become entangled (e.g., with the leaflets of mitral valve 14) during unprotected movement across the proximal-distal extent of the lumen of the left atrium. Advance of the device from the septum provides a potential advantage for reducing a chance of inadvertent puncture of the distal-side atrial wall while advancing overtube 110.

FIGS. 3C-3D shows the partially deployed cooling frame 101 from two different orientations. Cooling tube 102A is oriented generally to extend across the roof of the left atrium 10 (opposite the mitral valve 14). Cooling tube 102B extends across the dorsal wall of mitral valve 10. On dorsal wall 11, in positions within a loop area defined by the two cooling tubes 102A, 102B lie the ostia of the pulmonary veins 12A, 12B, 12C, 12D. Tensioning member 104 is partially deployed, but not fully activated to create pressure against ventral wall 13.

At block 212, in some embodiments, the deployed cooling frame 101 is pressed against the wall of the body organ lumen in which it is deployed. This is now explained further with respect to FIGS. 4A-4D.

Reference is now made to FIGS. 4A-4B, which schematically illustrate selected phases of the deployment of cooling frame 101 within a left atrium 10, according to some embodiments of the present disclosure. Further reference is made to FIGS. 4C-4D, which schematically illustrate expansion states of cooling frame 101 during deployment, corresponding to the in situ states described in relation to FIGS. 4A and 4B, respectively, according to some embodiments of the present disclosure.

FIGS. 4A and 4C represent the stage of deployment also shown in FIGS. 3C-3D. Main curve 105A of tensioning member 104 is deployed and awaiting further actuation to press the cooling tubes 102A, 102B of cooling frame 101 into position.

FIGS. 4B and 4D show the configuration of tensioning member 104 after it has been further extended out of overtube 110. The bulge of main curve 105A is increased, to the point where it contacts, and potentially stretches ventral wall 13. Secondary curve 105 optionally takes up some of the force, to avoid buckling of members of the cooling frame 101, and/or protect against damage to the walls of the atrium 10. The force of contact between main curve 105A and ventral wall 13 potentially acts to force cooling tubes 102A, 102B into position in substantially continuous contact with dorsal wall 11, surrounding (e.g., bracketing) the ostia of the pulmonary veins 12A, 12B, 12C, 12D.

Optionally, secondary curve 105 also performs a positioning function, e.g., by interaction with peripheral portions of mitral valve 14. This may help to force cooling frame 101 (and, e.g., cooling tube 102A in particular) upward against the roof of left atrium 10.

At block 214, in some embodiments, the deployed cooling frame 101 is activated to perform ablation. This is now explained further with respect to FIGS. 5A-5D.

Reference is now made to FIGS. 5A-5B, which schematically illustrate different positions of a coolant supply tube 120 within a cooling tube 120A of cooling frame 101, according to some embodiments of the present disclosure.

In some embodiments, cryoablation begins with coolant supply tube 120 located within cooling tube 102A, with its distal end 121 positioned in the vicinity of distal tip 106. Distal end, 121, in some embodiments, comprises an opening which acts as a supply port for coolant entry into the containment area of cooling tube 102A, 102B. Optionally, one or more supply ports are provided at other positions along cooling tube 102A, 102B; for example, at the distal end, proximal end, and/or near the middle of cooling tube 102A, 102B. For purposes of discussion, examples are presented in which distal end 121 acts as a supply port; however, other supply port positions and/or patterns (i.e., of two or more supply ports) along cooling tube 120 are optionally used.

Optionally, coolant discharges from distal end 121 to flow into both of cooling tubes 102A, 102B, e.g., back across its own longitudinal extent where it passes through cooling tube 102A, and across a lumen of tip 106 to flow back through cooling tube 102B. Additionally or alternatively, a coolant supply tube 120 is located within cooling tube 102B. If, for example, there is a coolant supply tube 120 feeding each of cooling tubes 102A, 102B, the two tubes optionally are not in fluid communication with each other.

The coolant delivered may be, for example, nitrogen. In some embodiments, another coolant is optionally used, for example, nitrox and/or argon. Delivery pressure is, optionally between 40-90 Bars (e.g., when using a liquid evaporation method of cooling), or a higher pressure (e.g., 150-400 Bars when using Joule-Thompson cooling). Coolant delivery tube 120 is optionally, e.g., about 300 μm in outer diameter, with a wall thickness of about 50 μm.

Coolant fluid leaving tube coolant supply tube 120 can create cooling according to one or more different mechanisms.

In adiabatic (Joule-Thompson) cooling, expansion of, e.g., nitrogen in a pressurized and/or liquid (but not necessarily cooled) state does work on its surroundings, causing it to lose heat energy and cool. If the coolant is delivered in liquid form, there may also be release of heat energy into expansion causing cooling as the coolant undergoes a phase change between liquid and gas. In either case, the larger internal diameter of cooling tubes 102A, 102B compared to coolant supply tube 120 will tend to allow significant expansion to occur at distal end 121, where coolant exits delivery tube 120.

Moreover, in some embodiments (particularly but not exclusively embodiments using Joule-Thompson effect cooling), the expansion-cooled fluid is passed back along the extent of coolant supply tube 120. This allows a certain amount of heat exchange cooling to take place, creating a feedback cycle. Gas travelling distally to be expanded exchanges heat with the already expansion-chilled return gas, cooling it. Then, when it reaches distal end 121 of the coolant supply tube 120, expansion cools it still further. This further lowers the temperature of the gas that passes back along coolant supply tube 120 and increases the amount of pre-chilling, until eventually a steady state of maximal cooling is potentially reached. Optionally, exchange surface area is increased, e.g., by coiling one or more of the return conduit and the coolant supply tube.

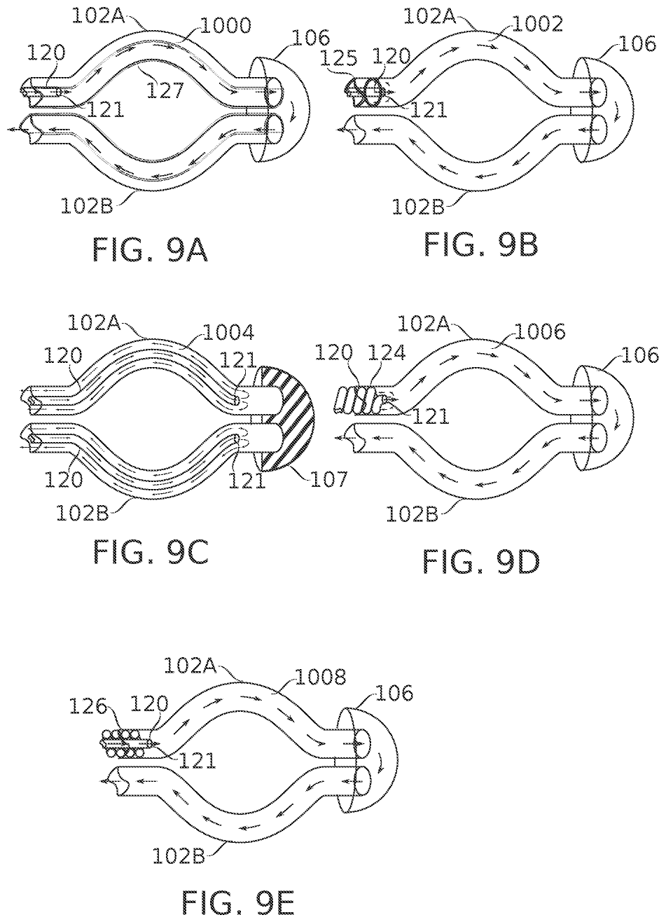

Cooling frame 101 is also used, in some embodiments, with an alternative arrangement for the delivery, distribution, and/or flow of coolant within cooling tubes 102A, 102B. Examples of such arrangements are discussed in relation to FIGS. 9A-9E, herein.

Optionally, coolant is delivered completely or to some extent pre-chilled from outside the device (e.g., below ambient temperature). Optionally, pre-chilled coolant is used for non-expansion cooling. However, as there is a relatively great distance to travel along overtube 110 before reaching cooling frame 101, and restrictions within a catheter on insulation thickness along that distance, this is potentially insufficient to establish cryoablation conditions on its own.

Further reference is made to FIG. 5C, which is a schematic flowchart of a method of delivering coolant to cooling frame 101, according to some embodiments of the present disclosure.

The flowchart of FIG. 5C begins with the cooling frame 101 already in position for cryoablation, for example as described in relation to FIG. 4B.

At block 610, in some embodiments, coolant is supplied through the coolant supply tube 120 into one or more of the cooling tubes 102A, 102B.

At block 612, in some embodiments, supply tube 120 slides (e.g., is advanced and/or retracted) through the one or more of the cooling tubes 102A, 102B, so that a delivery port for the cooling fluid (e.g., distal aperture 121) is moved to a new location.

In the embodiments illustrated by FIGS. 5A-5B, coolant supply tube 120 is optionally configured to be proximally withdrawn during cooling. This provides a potential advantage by allowing cooling frame 101 to be operated in a manner which focuses the coldest coolant first at a distal position on the cooling frame (near tip 106), and later in progressively more proximal regions (and/or conversely, focusing cooling first proximally then moving distally). It is a potential advantage to being cooling at an end (distal or proximal) of the device, since the ends also receive substantial mechanical support from, e.g., overtube 10 and/or tensioning member 104, and there may be a period of weakening support as the metal cools past the temperature range of its superelasticity. There is also a potential advantage for managing, e.g., by a rate of movement of coolant supply tube 120 the transition between the warmer, superelastic state of cooling tubes 102A, 102B and the cryogenically chilled, potentially non-superelastic state of cooling tubes 102A, 102B.

Optionally, movement of the distal end of coolant supply tube 120 includes passage through a channel region of distal tip 106, e.g., cooling in one cooling tube 102A proceeds from a proximal to a distal direction, then in the second cooling tube 102B from a distal to a proximal direction, as coolant supply tube 120 is withdrawn.

Reference is made to FIG. 5D, which schematically illustrates a two-tube arrangement for coolant supply, according to some embodiments of the present disclosure. In some embodiments, a separate coolant supply tube 120 is provided for each of cooling tubes 102A, 102B. An example is shown schematically in FIG. 5D, wherein cap 107 is constructed without a channel allowing fluid communication between the cooling tubes 102A, 102B. Distal ends of coolant deliver tubes 120 are shown superimposed in several positions 121A, 121B, 121C, 121D, 121E, 121F, illustrating how cooling can be focused at different positions along cooling tubes 102A, 102B. In some embodiments, cooling tube 120 has ports at a plurality of these locations; optionally, the ports can be moved, e.g., so that each port slides across a different portion of cooling tube 102A, 102B.

Returning to the discussion of FIG. 5C: it is a potential advantage, during cooling, that loss of structural strength associated with reductions in superelasticity be replaced by having the cooling tubes 102A, 120B frozen into place against (freezingly adhere to) the tissue they contact. There is potentially a period of vulnerability of good contact during the change in temperature—e.g., a period while superelasticity is weakened, but before frozen adherence has been established. It is a potential advantage to reduce the duration of this vulnerable period (e.g., by ensuring that regions transition quickly from warm to cold). In some embodiments, the shape memory transition temperature of the alloy used to make at least one of the cooling tubes (e.g., the temperature at which transformation completes, typically notated as A_{f}) is set to be near or below the freezing point of water and/or blood (about 0° C. or below), which potentially helps to minimize the chances that loss of strength will result in loss of good thermal contact with the lumenal wall.

It is noted that the progressive cooling method provides a potential advantage by focusing cooling power on relatively short lengths of tubing, allowing them to quickly transition from maintaining tissue contact by superelastic tension to maintaining tissue contact by frozen adherence. Furthermore, this is potentially achieved while other portions of a cooling tube 102A, 102B remain at least to some extent superelastic, potentially helping to maintain device stability.

In some embodiments, cooling is intensified particularly at one or more regions along a cooling tube 102A, 102B, for example by increasing cryogenic fluid flow and/or increasing dwell time for a delivery port of coolant supply tube 120 to gain a greater spread of.

The flowchart of FIG. 5C includes aspects and variations of coolant supply tube 120 and/or its movement; for example as described in relation to FIGS. 5A-5B and 5D, and/or FIGS. 9A-9E, herein.

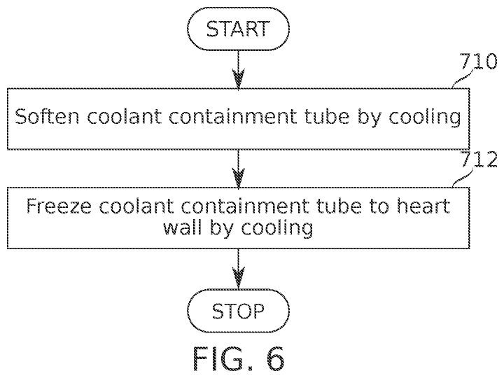

Reference is now made to FIG. 6, which is a schematic flowchart of a method of maintaining contact of a cooling frame with a heart during operation, according to some embodiments of the present disclosure.

The flowchart begin with the cooling frame 101 already in position for cryoablation, for example as described in relation to FIG. 4B. In particular, cooling tubes 102A, 102B are in surface contact with an internal surface of an organ lumen; for example, a left atrium, along a path which substantially surrounds one or more pulmonary vein ostia.

At block 710, in some embodiments, coolant is supplied through the coolant supply tube 120 into one or more of the cooling tubes 102A, 102B (cooling tubes 102A, 102B correspond to the cooling containment tubes mentioned in the block diagram text of FIG. 6). Cooling tubes 102A, 102B, in some embodiments, comprise a superelastic alloy which loses some or all of its superelastic properties as it reaches cryoablation temperatures.

At block 712, in some embodiments, one or more of cooling tubes 102A, 102B reaches a temperature cold enough to freeze surrounding water-containing liquid into ice, and potentially much lower (e.g., −40° or lower). At sufficiently low temperatures, ice formation (and consequently, freeze-adherence) may occur within seconds even in the presence of blood flow.

Optionally, the freezing occurs first at a position along cooling tube 102A, 102B which is radially outside the position of a supply port of coolant supply tube 120 (e.g., radially outside the position of distal end 121). This potentially helps to ensure that the first-softening region of coolant tube 102A, 102B is also be the first freeze-adhering portion of coolant tube 102A, 102B. This provides a potential advantage for shortening or eliminating the period during which neither contact-promoting mechanism is functioning effectively in that local region.

### Distal Tips of a Cooling Frame

Reference is now made to FIG. 7, which schematically illustrates a cutaway view of a channeled frame connector 107 at a distal tip 106 of cooling frame 101, according to some embodiments of the present disclosure.

In some embodiments, cooling tubes 102A, 102B are fluidly interconnected with one another at distal tip 106 via a channel 502 in cap 107. In some embodiments, cap 107 comprises a tapered end 506 (optionally blunted; or sharp as shown.) Cap 107 is optionally made, for example, of metallic and/or polymeric material, for example, polyether block amide. Cap 107 is optionally comprised of metal coated with polymeric material, for example, stainless steel coated with polytetrafluoroethylene (PTFE). Tensioning member 104 is optionally attached, e.g., to a third lumen of frame connector 107, or embedded during molding of frame connector 107. Optionally, tensioning member 104 is attached directly to one or more of cooling tubes 102A, 102B at a location proximal to frame connector 107.

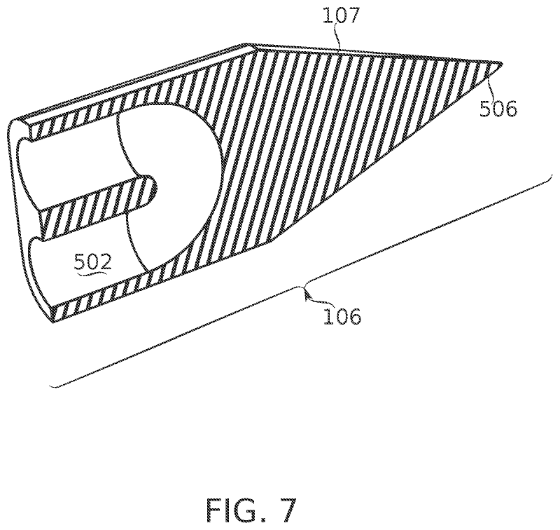

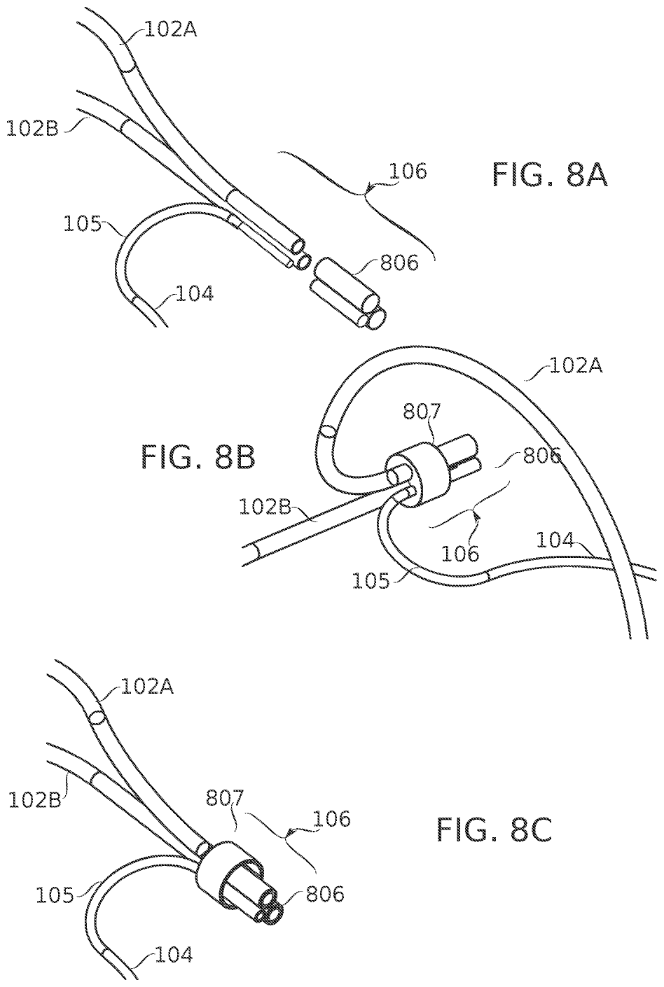

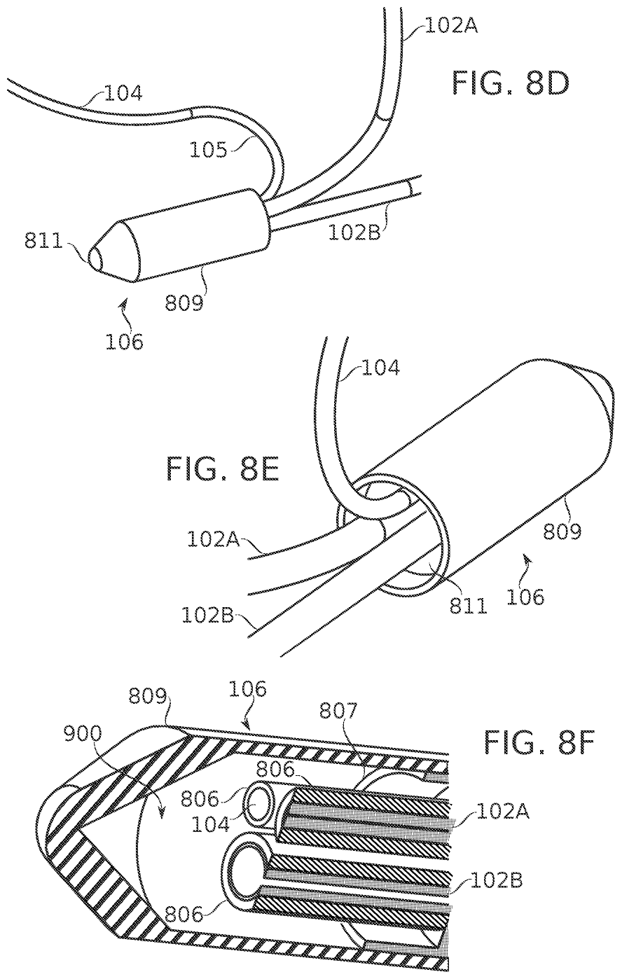

Reference is now made to FIGS. 8A-8F which illustrate stages in the manufacture of a frame connector placed at a distal tip 106 of a cooling frame 101, according to some embodiments of the present disclosure.

Nitinol can be a difficult metal to reform into a sealed enclosure, particularly one which is intended to withstand high pressures. FIG. 8A-8F explain a method of construction by means of which nitinol cooling tubes 102A, 102B are optionally bound into a leak-proof tip enclosure.

In some embodiments (FIG. 8A), the frame connector comprises a plurality of metal connecting tubes, optionally each attached to the other to form a sleeve assembly 806. Assembly of sleeve assembly 806 to cooling tubes 102A, 102B and tensioning member 104 comprises slipping over them and attaching; attaching is optionally by soldering and/or crimping. In some embodiments, connecting tubes 806 comprise a non-nitinol metal such as stainless steel, cobalt chrome, or another metal.

Added over sleeve assembly 806, FIGS. 8B-8C show cap 807. Cap 807, in some embodiments, is welded to sleeve assembly 806. Cap 807 may be filled, e.g., with epoxy, potentially increasing the stability and fixation of sleeve assembly 806 itself, as well as its connection with cap 807. It is noted that cooling tube 102A, 102B protrude past cap 807.

In some embodiments, hollow tip 809 is placed over cap 807 (e.g. slid over from a distal side; FIGS. 8D-8F). Tip 809 is closed on its distal side, and sealingly attaches over cap 806. Optionally, sealing comprises creation of a continuous laser welding line, and/or use of epoxy (e.g. additional filler material). Optionally, tip 809 terminates in a tapered end 811. Optionally, hollow tip 809 is comprised of a soft polymer, for example, polyether block amide.

In some embodiments, hollow tip 809 encloses a hollow chamber 900, through which cooling tubes 102A, 102B are in fluid communication. Alternatively, in some embodiments, cavity 900 is also filled (e.g., with epoxy), terminating cooling tubes 102A, 102B so that they are not in fluid communication with each other.

### Circulation Patterns of Coolant Within a Cooling Frame

Reference is now made to FIGS. 9A-9E, which represent different methods of circulating cooling fluid within a cooling frame 101, according to some embodiments of the present disclosure.

In FIG. 9A, coolant supply tube 120 is placed in a cooling tube 102A, which is in fluid communication with another cooling tube 102B, interconnected through distal tip 106. Cooling is optionally achieved by gas expansion and/or liquid evaporation as coolant exits one or more ports of coolant supply tube 120. Once coolant is delivered, the flow pattern 1000 draws coolant distally through cooling tube 102A, into tip 106, and then proximally out through cooling tube 102B. Optionally, coolant delivery tube 120 is movable within the tubes to change the position at which initial expansion occurs. Optionally, tube 120 is advanceable; optionally, tube 120 begins fully inserted (e.g., all the way forward and then bending around to reach back to a proximal area of cooling tube 102B), and is withdrawn during cooling to reach all parts of the cooling frame 101. In some embodiments, tube 120 can be alternately—optionally repeatedly—advanced and withdrawn. This has a potential advantage for increasing temperature uniformity.

Optionally, surface portions of cooling tube(s) 102A, 102B which are not used for transferring thermal energy from the lumen surface are provided with an insulating coating and/or lining. For example, a partial-circumferential coating 127 is optionally provided, as shown in FIG. 9A. The portion of the circumference insulated (inside and/or outside) may be about 30%, 50%, or 70%, for example. This potentially helps to increase the efficacy of cryoablation. It should be understood that this lining or coating is optionally applied to any of the cooling tube embodiments described herein.

FIG. 9B illustrates substantially the same configuration (admissible of the same variants), as FIG. 9A, except that in FIG. 9B, a portion of the coolant flow pattern 1002 directs coolant back proximally along coolant supply tube 120. This potentially creates counter-cooling, leading to a feedback cycle that may allow lower temperatures to be reached. Optionally, an insulating polymer lining 125 is provided within and/or over at least the portion of cooling tube 102A in which counter-cooling occurs.

FIG. 9C illustrates a variant where cap 107 connects but prevents fluid communication between cooling tubes 102A, 102B. Each cooling tube 102A, 102B has its own coolant supply tube 120. Circulation pattern 1004 separately extends proximally along the full length of both of cooling tubes 102A, 102B, with at least one supply port (e.g., distal end 121) located within the distal portion of each cooling tube 102A, 102B.

FIG. 9D illustrates a variant of the situation of FIG. 9B, wherein surface area for counter-cooling is increased by configuring a portion of coolant supply tube 120 in the form of a coil 124. Alternatively, in some embodiments, a coolant return tube 126 is arranged as a coil around coolant supply tube 120, for example as shown in FIG. 9E. In some embodiments, both the return path and the coolant supply tube 120 are arranged in coils, e.g., interdigitated coils.

Another arrangement, in some embodiments, is to flow cold fluid directly through the cooling tubes 102A, 102B, optionally without additional cooling at the site of the cooling frame 101.

### Other Frame Configurations

### Redoubling Cooling Tube

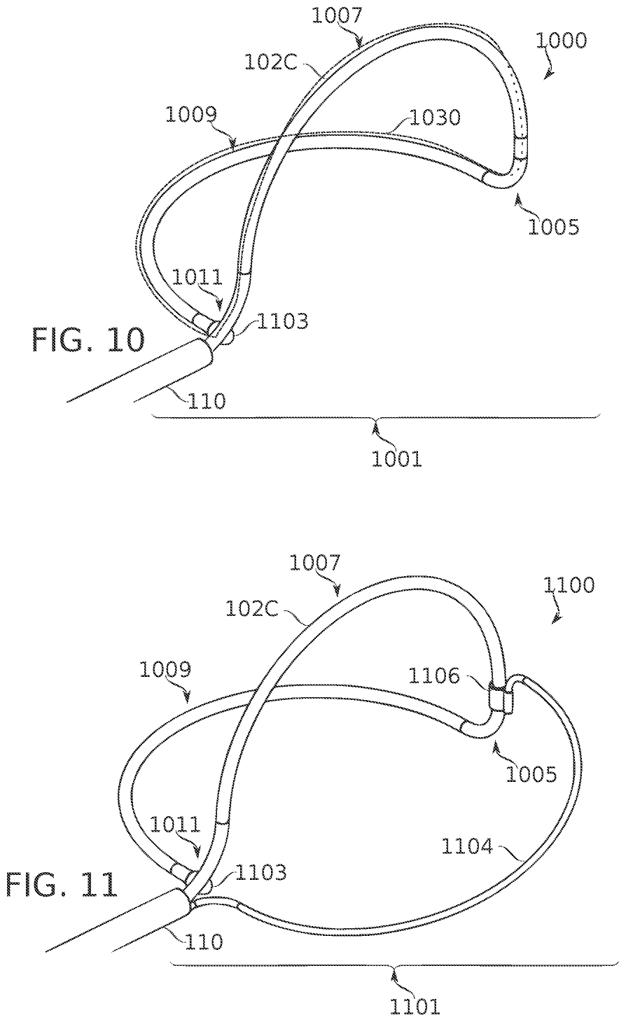

Reference is now made to FIG. 10, which schematically represents a cooling frame 1001 of a cryoablation catheter 1000 comprising a redoubling cooling tube 102C, according to some embodiments of the present disclosure. Reference is also made to FIG. 11, which schematically represents a cooling frame 1101 of a cryoablation catheter 1100 comprising a redoubling cooling tube 102C and a tensioning member 1104, according to some embodiments of the present disclosure.

In some embodiments, a cooling frame 1001, 1101 comprises a redoubling cooling tube 102C. In its constrained and collapsed form (e.g., while still confined within overtube 110), cooling tube 102C extends in a straightened configuration from a proximal to distal direction, terminating in a tube cap 1103.

Cooling tube 102C, in some embodiments, comprises a superelastic, shape-memory alloy such as nitinol. Upon advancement distally from the overtube 110, the cooling tube 102C assumes a redoubled configuration. The redoubled configuration extends distally (e.g., in an arc 1007, optionally a planar arc) to distal bend 1005, changes direction at distal bend 1005 and re-curves proximally (e.g., in another arc 1009, optionally a planar arc); returning to meet itself near its own proximal side 1011. Optionally, it meets itself at about the place where it exits overtube 110. The overall deployed shape of cooling frame 1001, 1101 defines a contact surface (underneath loop 1030), shaped to be pressed into contact with tissue of the curved interior surface of a target organ lumen. The contact surface underneath loop 1030 is, for example, substantially as described, for example, in relation to loop 130 of FIG. 1A. As also for the contact surface indicated by loop 130, actual breaks in continuity of contact (for example, at proximal side 1011) are potentially overcome by the spread of lesioning during cryoablation, e.g., to a distance of 1-5 mm or more from regions of direct contact.

The cooling frame 1001 of FIG. 10 is shown without a tensioning member. Instead, cooling frame 1001 relies on the intrinsic shape memory and elasticity of cooling tube 102C to achieve contact with the interior lumenal surface of the target organ lumen.

In some embodiments (FIG. 11), tensioning member 1104 is provided. Tensioning member 1104 potentially increases a reliability of surface contact of cooling frame 1101, compared to cooling frame 1001. Tensioning member 1104 has a distal extension distance from overtube 110 separately controllable from the distal extension of cooling tube 102C, for example, similar to the operation to tensioning member 104. Tensioning member 1104 connects at its distal end to connector 1106. Connector 1106 is placed near the distal-most position of redoubled cooling tube 102C, for example, at about the position of distal bend 1005, e.g., adjoining one side of distal bend 1005. This position is also near the middle of cooling tube 102C, for example, when cooling frame 1101 is in its collapsed state. Optionally connector 1106 comprises a plurality of short stainless steel tubes. The tubes may be welded to each other, and, for example, crimped and/or adhered to the cooling tube 102C and tensioning member 1104. Optionally, most (e.g., through at least 80% or 90% of its length) of tensioning member 1104 extends through a planar arc. Optionally, tensioning member 1104 connects to connector 1106 from a direction which is on the proximal side of connector 1106, at least when the cooling frame is in its collapsed (substantially linear) state. This potentially means that tensioning member 1104 does not need to pass through an extremely tight (e.g., 4 mm or less) radius of curvature when packaged. Such a tight radius of curvature would potentially increase a risk of device failure, and/or create difficulties for reliable manufacture.

It should be noted that the shape of the redoubled tube is not necessarily limited to the shape shown. For example, the arcs of the redoubled tube are optionally non-planar, undulating, and/or helical or partially helical.

### Single-Arc Cooling Frame

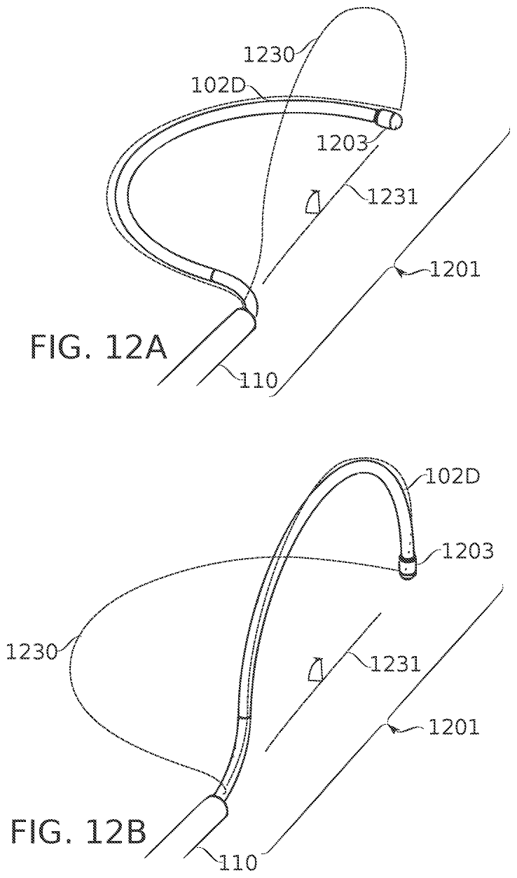

Reference is now made to FIGS. 12A-12B, which schematically illustrate a cooling frame 1201 comprising a single lumen-spanning arc of a single cooling tube 102D, according to some embodiments of the present disclosure.

In some embodiments, a single-arc cooling frame 1201 is provided. To ablate a whole loop (e.g., of a lumen surface extending substantially along loop 1230), the cooling tube 102D is operated sequentially in two different positions. For example, FIG. 12A shows cooling tube 102D in a first position for cooling, and FIG. 12B shows cooling tube 102D rotated (e.g., around axis 1231) and placed in a second position for cooling. In the first and second positions, distal and proximal sides of cooling tube 102D remain in about the same positions, so that a substantially closed loop is formed by cryoablation. The proximal side, for example, is near an exit from overtube 110, and the distal side may be near distal cap 1203. The ablation order for the two positions is optionally first position, then second position; or the reverse.

### Swiveling, Single-Arc Cooling Frame

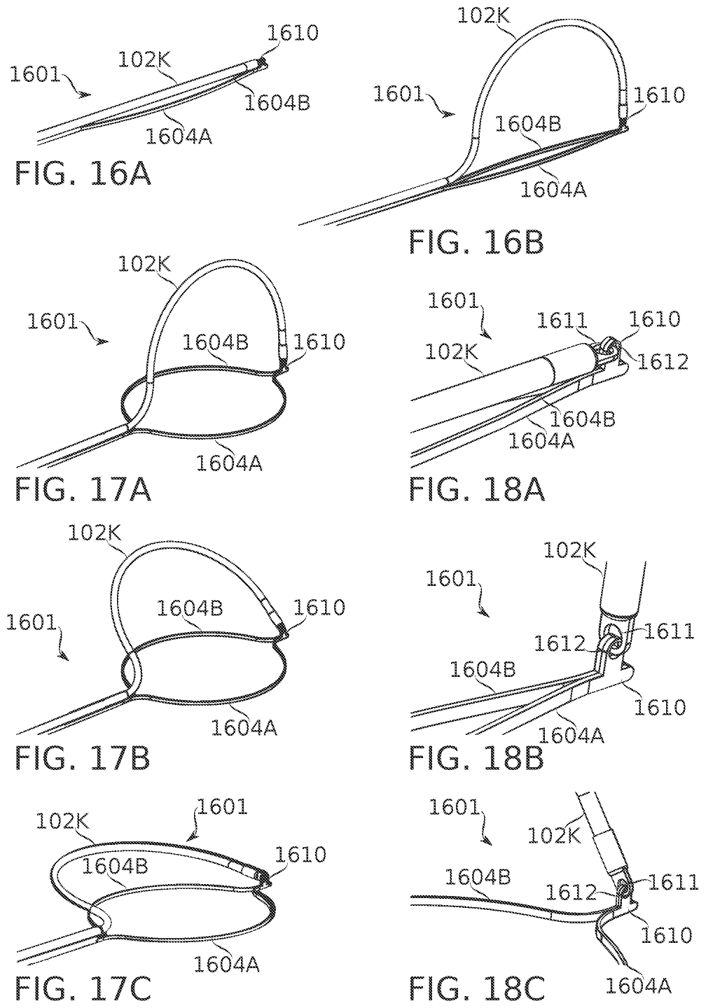

Reference is now made to FIGS. 16A-18C, which schematically illustrate a cooling frame 1601 comprising a swiveling distal connection 1610, according to some embodiments of the present disclosure.

In some embodiments, a cooling frame 1601 comprises a tensioning element 1604A, 1604B which is connected to a distal end of a cooling tube 102K. In some embodiments, cooling frame 1601 is a single cooling tube design.

Tensioning element 1604A, 1604B, in some embodiments, comprises two arcs which expand oppositely upon deployment to anchor substantially around a circumference of a lumen targeted for ablation. Thereby, cooling frame 1601 provides an anchor (the region of swiveling distal connection 1610) which remains substantially in place while allowing separate manipulation of cooling tube 102K. This provides a potential advantage for reliability and/or stability of placement of cooling tube 102K. For example, cooling tube 102K can be operated to ablate in a first position, and then in a second position, while assuring that its distal end remains positioned in a same region so that the loop of a cryoablation lesion will be closed.

FIG. 16A shows the cooling frame pre-expansion (e.g., collapsed for delivery, as it would be while confined within an overtube 110, not shown in this drawing). Upon distal advance of cooling frame 1601 from overtube 110 (FIG. 16B and then FIG. 17A), tensioning element portions 1604A, 1604B expand away from each other to create a loop-shaped anchor.

Along with this (although optionally separately controllable), cooling tube 102K advance distally to take up an arc-shaped configuration. In some embodiments, the components are biased toward their expanded configurations, e.g., by the use of a superelastic and shape memory metal alloy such as nitinol. In some embodiments, advancement from the proximal side while holding the distal end in position forces components to expand.

Once the cooling frame 1601 is deployed, cooling tube 102K can be moved to different positions (e.g., as shown in FIGS. 17A-17C) in order to perform cryoablation. In some embodiments, moving to a new position comprises pulling cooling tube 102K slightly proximally to un-expand it (e.g., after a first cryoablation), rotating cooling tube 102k (e.g., by rotation of an external control member), then re-expanding the cooling tube 102K by pushing it distally again. Once re-positioned, a second cryoablation may be performed.

Stability of the position of the proximal end is assured by maintaining a position of the overtube 110, while stability of the distal end is assured by maintaining a position of the expended tensioning element 1604A, 1604B.

FIGS. 18A-18C illustrate details of swiveling distal connection 1610. In some embodiments, swiveling distal connection 16010 comprises two interlocking loops 1611, 1612. The loop connection allows movements around two different rotational axes. In the first movement, cooling tube 102K is free to rotate approximately through 90° from a flattened configuration (FIG. 18A) to a deployed, arc-shaped configuration (FIG. 18B). In the second movement, the deployed cooling tube 102K is rotatable, e.g., through the positions shown in FIGS. 17A-17C. The range of movement allowed around for this axis of rotation optionally comprises at least 45° of rotation, and optionally 90° or more of rotation.

While uses of a single-arced cryoablation frame using two-position ablation protocol has just been described, it should be noted that the single arc is optionally used for ablation in only one position (e.g., to supplement and/or correct results of another ablation procedure), and/or in three or more positions.

### Unconnected-Arc Cooling Frames

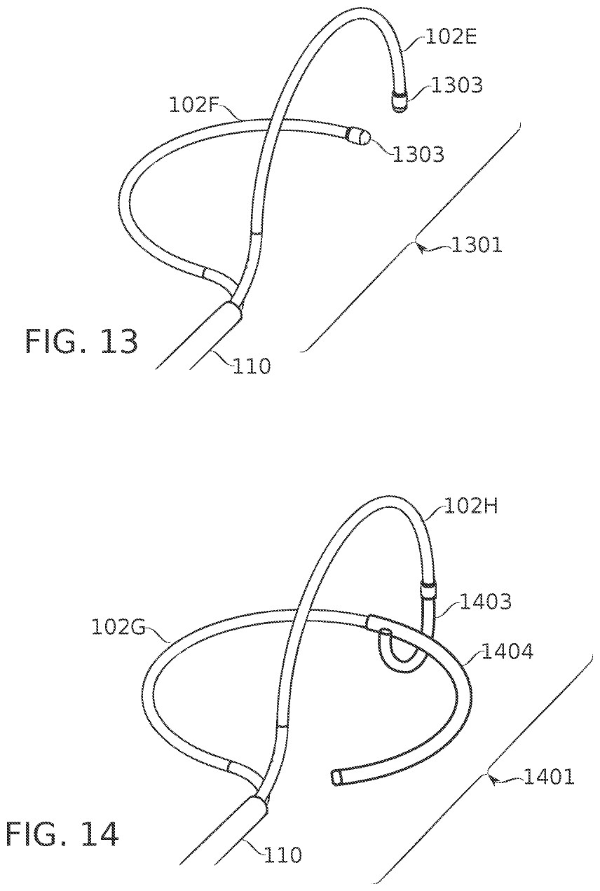

Reference is now made to FIG. 13, which schematically illustrates a cooling frame 1301 comprising two separate lumen-spanning arcs comprising cooling tubes 102E, 102F, respectively, according to some embodiments of the present disclosure.

In some embodiments, cooling tubes 102E, 102F extend separately through their arcs from a distal side near where they exit overtube 110 to their respective distal caps 1303. The cooling tubes 102E, 102F again comprise a superelastic and shape memory alloy such as nitinol. When unconnected, there is potentially less certainty of position, continuous lumenal surface contact, and/or loop closure, however tube positioning can be verified and/or adjusted, for example, under fluoroscopic examination.

Reference is now made to FIG. 14, which schematically illustrates a cooling frame 1401 comprising two separate lumen-spanning arcs comprising cooling tubes 102G, 102H, respectively, each having its own tensioning element 1403, 1404, according to some embodiments of the present disclosure.

In some embodiments, at least one of cooling tubes 102G, 102H is provided with its own tensioning element 1403, 1404. The design of the tensioning element can be adjusted, depending on the relevant geometry of the target lumen. For example, tensioning element 1404 substantially continues the arc of cooling tube 102G, allowing it to create force by pressing against an opposite wall of the target lumen than that contacted by cooling tube 102G. Additionally or alternatively, tensioning element 1403 curves to create a blunted end at a position near to the distal wall of the lumen, where it potentially operated by pressing against structures at or near to the distal wall, such as tissues comprising a ring of a mitral valve.

Optionally, the tensioning elements 1403, 1404 are separately extendable, e.g., slideable over their respective cooling tubes 102G, 102H. Optionally, they are of fixed length, and extend along with their respective cooling tube

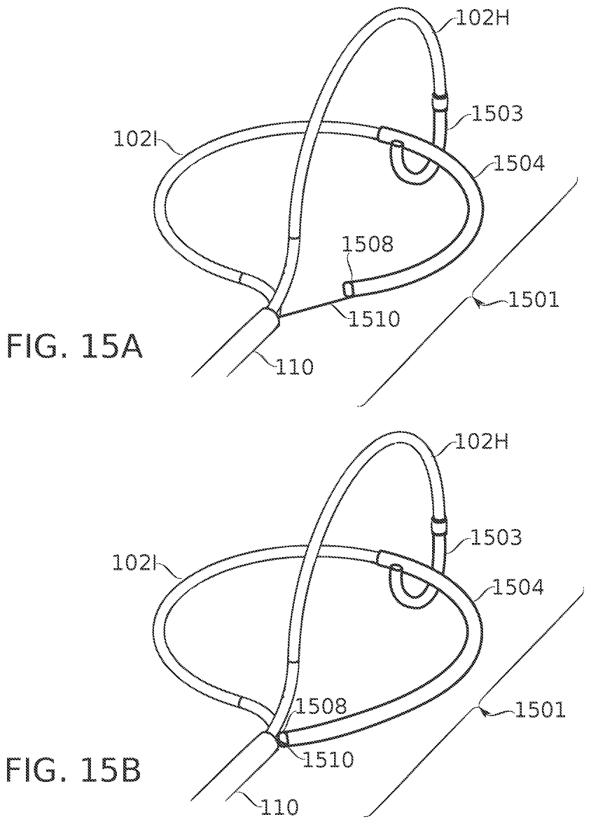

Reference is now made to FIGS. 15A-15B, which schematically illustrate a cooling frame 1501 comprising at least one shaping member 1510, which is operable to pull a free distal end 1508 of a cooling tube 1021 and/or of an extension 1504 of the cooling tube, back toward a proximal region of the cooling frame 1501. Potentially, this helps stabilize deployment of the cooling frame 1501. The cooling frame, in some embodiments, comprises any configuration having a free end extending beyond the distal side of the cooling frame, for example, the configuration of FIGS. 10-11 (wherein cooling tube 102C itself terminates the free distal end), or, as illustrated, the configuration of FIG. 14 (wherein a tensioning member 1404 terminates the distal free end).

In some embodiments, shaping member 1510 comprises a wire. Shaping member 1510 is allowed to be pulled from overtube 110 by extrusion of cooling tube 102I. To complete deployment, shaping member 1510 is then shortened (pulled proximally) again, drawing free distal end 1508 back toward the proximal side of the cooling frame.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

In addition, any priority document(s) of this application is/are hereby incorporated herein by reference in its/their entirety.

1. A cooling frame of a cryoablation catheter comprising:
a proximal side;
a distal connector, sized to fit within an overtube of a catheter;
tubing defining at least one extent of cooling tube configured to be chilled by a cooling flowing therein, and extending between the proximal side and the distal connector region; and
a tensioning strut, extending between the proximal side and the distal connector.

2. The cooling frame of claim 1, wherein the cooling frame comprises at least a second extent of cooling tube extending between the proximal side and the distal connector region.

3. The cooling frame of claim 1, wherein the cooling frame is configured to self-expand from a collapsed configuration sized to fit within the catheter overtube to an expanded configuration.

4. The cooling frame of claim 3, wherein the cooling frame comprises at least a second extent of cooling tube extending distally from the distal connector region in the collapsed configuration, and, in a deployed configuration, recurving from the distal connector in a proximal direction back to the proximal side of the cooling frame.

5. The cooling frame of claim 1, wherein a deployment length of the tensioning strut is configured to be advanced relative to the catheter overtube separately from the cooling tubing while remaining connected to the cooling tubing at the distal connector.

6. The cooling frame of claim 1, wherein the at least two extents of cooling tubing deploy by assuming a curvature that defines an ablation line configured to be brought into contact with a targeted isolation region.

7. (canceled)

8. The cooling frame of claim 6, wherein a main curve of the tensioning strut deploys by radial expansion away from central proximal-to-distal axis of the cooling frame in a direction away from the ablation line.

9. The cooling frame of claim 1, wherein the cooling frame is sized to deploy within a left atrium lumen, with a region of lumenal wall comprising the pulmonary vein ostia located between contacts of the two extents of cooling tubing with lumenal tissue of the left atrium, and the tensioning strut positioned radially opposite the region of lumenal wall comprising the pulmonary vein ostia.

10. The cooling frame of claim 1, wherein the main curve of the tensioning strut has an anisotropic cross-section at least 1.5× longer in a first direction than in a direction perpendicular to the first direction.

11-12. (canceled)

13. The cooling frame of claim 1, wherein the main curve expands to lie within a plane.

14. The cooling frame of claim 10, wherein the tensioning strut comprises a secondary curve, curving in a direction opposite the main curve.

15. The cooling frame of claim 14, wherein the secondary curve and the main curve lie substantially within a single plane.

16. The cooling frame of claim 14, wherein the main curve extends at least 70% of the way between the proximal side and the distal connector, when the cooling frame is deployed, and the secondary curve extends the remainder of the way to the distal tip.

17. The cooling frame of claim 1, wherein the wherein each of the at least one extents and the tensioning strut connect to a proximal side of the distal tip.

18. (canceled)

19. The cooling frame of claim 1, wherein at least one of the tubing and the tensioning strut comprises nitinol alloy.

20. (canceled)

21. The cooling frame of claim 1, comprising at least one coolant delivery tube, positioned in fluid communication with a lumen of the tubing, and configured to deliver coolant to the lumen; wherein a supply port of the coolant delivery tube is configured to move within the lumen of the tubing.

22. (canceled)

23. The cooling frame of claim 21, wherein the at least one coolant delivery tube comprises a plurality of supply ports configured to delivery coolant to the lumen.

24. The cooling frame of claim 21, configured with a lumenal region between the coolant deliver tube and the cooling tube, allowing return of coolant proximally past the coolant delivery tube, thereby creating a counter-cooling effect.

25. The cooling frame of claim 1, wherein the distal connector comprises a swivel joint, and comprising a plurality of tensioning struts extending between the proximal side and the distal connector.

26-30. (canceled)

31. The cooling frame of claim 2, wherein the at least two extents of tubing comprise a plurality of tubing pieces, each terminating distally at the distal connector, and the distal connector is a distal tip.

32-40. (canceled)
